(19) United States
(12) Patent Application Publication
(10) Pub. No.: US 2023/0201354 A1
(43) Pub. Date: Jun. 29, 2023

(54) CARBON NANOTUBES AND COMPLEXES THEREOF FOR TREATING AND DETECTING OCULAR TUMORS

(71) Applicant: THE REGENTS OF THE UNIVERSITY OF MICHIGAN, Ann Arbor, MI (US)

(72) Inventors: Hakan Demirci, Ann Arbor, MI (US); Nicholas Kotov, Ann Arbor, MI (US); Yichun Wang, Ann Arbor, MI (US)

(21) Appl. No.: 17/998,761

(22) PCT Filed: May 14, 2021
(86) PCT No.: PCT/US2021/032454

§ 371 Date: Nov. 14, 2022

Related U.S. Application Data
(60) Provisional application No. 63/025,768, filed on May 15, 2020.

(51) Int. Cl. A61K 47/52; A61K 9/00; A61K 45/06; A61K 49/00; A61K 47/69; C01B 32/159

(52) U.S. Cl. CPC A61K 47/52; A61K 9/0019; A61K 45/06; A61K 9/0092; A61K 49/0095; A61K 49/0017; A61K 47/6929; C01B 32/159; C01B 2202/36; C01B 2202/34; B82Y 5/00

(57) ABSTRACT

Disclosed herein are compositions and methods for injecting compounds into a vitreous body. Carbon nanotubes can be functionalized with a variety of agents, such as therapeutic agents and/or diagnostic agents, which can be injected into a vitreous body for treatment or detection of ocular tumors such as retinoblastoma. The carbon nanotubes can effectively penetrate the ocular tumor, making them effective carriers for the therapeutic and/or diagnostic agents.

### CROSS-REFERENCE TO RELATED APPLICATIONS

This application claims priority to U.S. Provisional Patent Application No. 63/025,768, filed on May 15, 2020, the entire contents of which are fully incorporated herein by reference.

### STATEMENT REGARDING FEDERALLY SPONSORED RESEARCH

This invention was made with government support under 1120923 and 1463474 awarded by the National Science Foundation, and W911NF-10-1-0518 awarded by the U.S. Army Research Office. The government has certain rights in the invention.

### BACKGROUND

Retinoblastoma is the most common intraocular cancer of childhood, accounting for about 2% of all childhood cancers. Within the last decade, the use of intraarterial and intravitreal chemotherapies has significantly changed the management of retinoblastoma. However, one problem with intravitreal injections is that medications might not fully penetrate the intraocular layers or tumors. Improved systems are needed for drug delivery to treat retinoblastoma and other ocular tumors.

### SUMMARY

Disclosed herein are compositions and methods for injecting compounds into a vitreous body. Carbon nanotubes can be functionalized with a variety of agents, such as therapeutic agents and/or diagnostic agents, which can be injected into a vitreous body for treatment or detection of ocular tumors such as retinoblastoma and uveal melanoma. The carbon nanotubes can effectively penetrate the ocular tumors, making them effective carriers for the therapeutic and/or diagnostic agents.

Carbon nanotubes (CNTs) are tubular, hollow nanostructures that have been utilized in biomedical applications including implantable devices. CNTs are biocompatible, and in vivo and in vitro studies have shown that CNTs have efficient drug-loading capacity with high length/diameter ratio and multifunctional surface chemistry (Tang et al. Adv. Mater. 2006, 18(24), 3203-3224; Gheith et al. Adv. Mater. 2006, 18, 2975-2979; Mohajeri et al. J. Cell Physiol. 2018, 234, 298-319; Yang et al. Curr. Drug Metab. 2012, 13, 1057-1067).

In the realm of biomedical applications, nanotubes and composites thereof have been used in implantable bioelectronic devices (Gheith et al. Adv. Mater. 2006, 18(22), 2975-2979; Shim et al. Nano Lett. 2007, 7(11), 3266-3273; Pappas et al. Nano Lett. 2007, 7(2), 513-519; Jan et al. Nano Lett. 2009, 9(12), 4012-4018). Carbon nanotubes have also been implemented for ex vivo drug delivery in various settings, but not in ocular tissues (Blanco et al. Curr. Opin. Chem. Biol. 2005, 9(6), 674-679; Zhang et al. Nanoscale Res. Lett. 2011, 6(1), 555).

### BRIEF DESCRIPTION OF THE DRAWINGS

FIG. 1A-1B show images of mouse eyes injected with functionalized carbon nanotubes. (A) From left to right, eyes injected with CNT-FITC-Bio days 1, 2, 3 and control eye (×10, DAPI). (B) From left to right, eyes injected with CTN-FITC-FA days 1, 2, 3 and control eye (×10, DAPI). The blue squares in each image denote the area in glass region, which is used for normalization. The red squares were used to calculate the averaged intensity within the tumor. FITC-functionalized CNTs appear as bright spots throughout the retinoblastoma tumor in colored images.

FIG. 2 shows the change of average intensity of CNTs functionalized with fluorescein isothiocyanate and biotin (“BIO medicine”), and fluorescein isothiocyanate and folic acid (“FA medicine”), over time.

### DETAILED DESCRIPTION

The present disclosure relates to compositions and methods for delivering therapeutic and/or diagnostic agents to ocular tumors, such as retinoblastoma tumors. As shown herein, carbon nanotubes (CNTs) with and without surface modification serve as effective carriers into the tumors following intravitreal injection.

Previous data regarding biodistribution of CNTs was obtained using systemic administration in animal models (Singh et al. Proc. Natl. Acad. Sci. U. S. A 2006, 103, 3357-3362; Sager et al. Nanotoxicology 2014, 8, 317-327). One study used a three-dimensional (3D) hepatocellular carcinoma tissue culture, and showed that surfaces of CNTs can be engineered to enable deep and fast penetration into certain tissues (Wang et al. ACS Nano 2015, 9, 8231-8238). However, the eye is a unique organ given its anatomical location and globe-shaped structure, with the vitreous cavity inside and retina/choroid layers on the wall. Data presented herein demonstrate that carbon nanotubes can penetrate through a retinoblastoma tumor when injected intravitreally, as shown in a transgenic retinoblastoma mouse model. The combination of deep penetration into the tumor with the ability of CNTs to carry active agents, such as anticancer drugs and diagnostic agents, provide systems and methods for diagnostics and treatment of hard-to-reach intraocular tumors. Data are presented herein for retinoblastoma tumors in a mouse model, and the methods are also applicable to other ocular tumors.

Accordingly, disclosed herein are compositions and methods of use thereof, the compositions comprising a complex of a carbon nanotube and a therapeutic agent (e.g., a chemotherapeutic agent) or a diagnostic agent.

### Carbon Nanotubes and Complexes Thereof

The carbon nanotubes can be single-walled, double-walled, or multiwalled. In some embodiments, the carbon nanotubes are single-walled carbon nanotubes (SWCNTs). In some embodiments, the carbon nanotubes are multi-walled carbon nanotubes (MWCNTs). Carbon nanotubes are commercially available from a variety of sources, for example, Sigma-Aldrich (St. Louis, Mo.) and Carbon Solutions, Inc. (Riverside, Calif.). Carbon nanotubes can be prepared by methods known in the art such as chemical vapor deposition, electric arc discharge, laser ablation, and high-pressure carbon monoxide disproportionation.

The carbon nanotubes have diameters from less than 1 nm up to about 100 nm. For example, in some embodiments the carbon nanotubes have an average diameter of about 0.5 nm, about 0.6 nm, about 0.7 nm, about 0.8 nm, about 0.9 nm, about 1.0 nm, about 1.1 nm, about 1.2 nm, about 1.3 nm, about 1.4 nm, about 1.5 nm, about 1.6 nm, about 1.7 nm, about 1.8 nm, about 1.9 nm, about 2.0 nm, about 2.5 nm, about 3.0 nm, about 3.5 nm, about 4.0 nm, about 4.5 nm, about 5.0 nm, about 5.5 nm, about 6.0 nm, about 6.5 nm, about 7.0 nm, about 7.5 nm, about 8.0 nm, about 8.5 nm, about 9.0 nm, about 9.5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, or about 100 nm. In some embodiments, the carbon nanotubes have an average diameter of about 0.5 nm to about 20 nm, about 0.5 nm to about 10 nm, about 0.5 nm to about 5 nm, about 0.5 nm to about 2.5 nm, or about 1.0 nm to about 2.0 nm.

The carbon nanotubes have lengths of about 50 nm to about 5000 nm, or about 100 nm to about 2500 nm, or about 100 nm to about 1500 nm. In some embodiments, the carbon nanotubes have an average length of about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, or about 1500 nm.

In some embodiments, the complex comprises a carbon nanotube and a therapeutic or diagnostic agent, in which the agent is non-covalently bound to the carbon nanotube. In some embodiments, the agent is adsorbed on the surface of the carbon nanotube, driven by non-covalent interactions such as van der Waals forces, hydrophobic interactions, π-π interactions, CH-π interactions, or the like. In other embodiments, the carbon nanotube is functionalized with a moiety that facilitates an ionic interaction or a hydrogen bonding interaction with the agent. For example, in some embodiments, the carbon nanotube is functionalized with a carboxylate group that interacts with a positively charged group on the agent (e.g., an ammonium group). As another example, the carbon nanotube is functionalized with a hydroxyl group that interacts via a hydrogen-bonding interaction with a suitable group on the agent (e.g., a carbonyl group). In some embodiments, preparation of such complexes starts from a carbon nanotube that is functionalized with the appropriate group (e.g., a carboxylate group or hydroxyl group), which may be commercially available or can be synthesized by methods known to those skilled in the art.

In some embodiments, the complex comprises a carbon nanotube and a therapeutic or diagnostic agent, in which the agent is covalently bound to the carbon nanotube. In some embodiments, preparation of such complexes starts from a carbon nanotube that is functionalized with a reactive moiety, such as a carboxylate group, a hydroxyl group, an amine group, a sulfhydryl group, an azide, an alkyne, or the like. These functionalized nanotubes may be commercially available or can be synthesized by techniques known in the art. Reaction with an agent (e.g., a therapeutic or diagnostic agent) bearing a complementary functional group results in covalent attachment of the agent to the carbon nanotube. For example, as those skilled in the art will appreciate, a carbon nanotube functionalized with a carboxylate group can react with a primary amine to form an amide bond (e.g., using a coupling agent such as a carbodiimide, e.g., DCC or CMC, or by first converting the carboxylic acid to an activated ester such as a succinimidyl ester). In some embodiments, the agent is directly attached to the functionalized carbon nanotube. In some embodiments, the agent is attached to the functionalized carbon nanotube via a linker.

In some embodiments, such as those in which the complexes are to be used in a method of treatment of an ocular tumor such as retinoblastoma, the complex includes a carbon nanotube and a therapeutic agent. Any suitable therapeutic agent may be used. In some embodiments, the therapeutic agent is a chemotherapeutic agent. For example, the therapeutic agent may be a chemotherapeutic agent identified on the “A to Z List of Cancer Drugs” published by the National Cancer Institute. In some embodiments, the therapeutic agent is an immunotherapeutic agent. In some embodiments, the therapeutic agent is a gene targeting agent, a protein kinase inhibitor, or a small molecule. In some embodiments, the therapeutic agent is a chemotherapeutic agent selected from the group consisting of carboplatin, cisplatin, cyclophosphamide, doxorubicin, etoposide, melphalan, topotecan, and vincristine. In some embodiments, the chemotherapeutic agent is selected from the group consisting of carboplatin, etoposide, and vincristine.

In some embodiments, the carbon nanotube is functionalized with more than one therapeutic agent, such as two or three different chemotherapeutic agents.

In some embodiments, such as those in which the complexes are to be used in a method of detecting of an ocular tumor (e.g., retinoblastoma), the compositions described herein include a complex of a carbon nanotube and a diagnostic agent. The diagnostic agent includes a group that is detectable, either directly or indirectly, by methods such as spectroscopic, photochemical, biochemical, chemical, or other methods. For example, useful detectable moieties include fluorophores, chromophores, luminophores, biotin, radioactive compounds, and imaging agents (e.g., contrast agents) used in positron emission tomography (PET), computed tomography (CT), single photon emission computerized tomography (SPECT), magnetic resonance imaging (MRI), terahertz or sub-terahertz spectroscopy with or without circular dichroism contrast (see, e.g., Choi et al. Nat. Mater. 2019, 18, 820-826), and the like. In some embodiments, the diagnostic agent directly generates a measurable signal, such as a fluorescent, chromogenic, luminescent, metallic, or radioactive signal. In some embodiments, the diagnostic agent is a fluorophore. In some embodiments, the diagnostic agent is a terahertz or sub-terahertz contrast agent. In other embodiments, the diagnostic agent includes a moiety for binding of a separate compound that generates the measurable signal (e.g., when the diagnostic agent is biotin, which can bind to a labeled streptavidin). In some embodiments, terahertz imaging will not require a contrast agent because the carbon nanotubes provide sufficient contrast.

In some embodiments, the diagnostic agent is a fluorophore. Suitable fluorophores include, but are not limited to, fluorescein and fluorescein dyes (e.g., fluorescein isothiocyanate or FITC, naphthofluorescein, 4′,5′-dichloro-2′,7′-dimethoxy-fluorescein, 6-carboxyfluoresceins (e.g., FAM)), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, SPECTRUM RED™, SPECTRUM GREEN™, cyanine dyes (e.g., CY-3™, CY-5™, CY-3.5™, CY-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. Examples of suitable fluorescent dyes that can be used and methods for linking or incorporating fluorescent dyes to other compounds (e.g., carbon nanotubes) can be found in The Molecular Probes Handbook— A Guide to Fluorescent Probes and Labeling Technologies, Molecular Probes, Eugene, Oreg., ThermoFisher Scientific, 11th Edition. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Beverly, Mass.).

In some embodiments, the carbon nanotubes have other surface modifications (covalent or non-covalent) e.g., to enhance certain properties such as solubility or processability. For example, carbon nanotubes can be functionalized (covalently or non-covalently) with polymers. Exemplary polymers include, but are not limited to, polyalkylene glycols (e.g., polyethylene glycol, polypropylene glycol, or copolymers thereof), biopolymers (chitosan, sodium alginate, cellulose derivatives, and the like), polyesters (e.g., polylactic acid, polyglycolic acid, poly(lactic-co-glycolic acid), and copolymers thereof. Carbon nanotubes can also be functionalized (covalently or non-covalently) with proteins such as streptavidin, fibronectin, bovine serum albumin, and the like. Surfactants can also be used to modify carbon nanotubes; exemplary surfactants include sodium dodecyl sulfate, sodium dodecylbenzenesulfonate, cetyltrimethylammonium bromide, Triton® X-Series surfactants, polyoxyethylene sorbitan monooleate, and similar surfactants. Other surface modifications include nanoparticles, DNA, sugars, and lipids.

Carbon nanotubes can also be functionalized with targeting ligands. For example, the folate receptor is upregulated in a variety of human cancers, and therefore folic acid can be complexed to a carbon nanotube to facilitate uptake of the carbon nanotubes into cancer cells (e.g., retinoblastoma cells). Retinoblastoma cells also have a specialized high-affinity carrier-mediated system for biotin uptake, and biotin therefore may also be used as a ligand to target retinoblastoma cells. Other targeting ligands such as proteins, antibodies, peptides, metabolites, and receptors can also be used. The targeting ligands can be specific for retinoblastoma or other ocular tumors.

The carbon nanotubes can also be functionalized with magnetic particles. This will enable magnetic guidance through ocular tissues, which can be concomitantly monitored by optical spectroscopy.

The carbon nanotubes can also be modified with multifunctional nanoparticles, using a glutaraldehyde cross-linking procedure (see, e.g., Mamedova et al. Nano Lett. 2001, 1(6), 281-286; Wang et al. Nano Lett., 2002, 2(8), 817-822).

### Pharmaceutical Compositions

Disclosed herein are pharmaceutical compositions for intravitreal injection, comprising a complex of a carbon nanotube and a therapeutic (e.g., chemotherapeutic) or diagnostic agent, and one or more pharmaceutically acceptable excipients. The term “excipient” is used herein to describe an ingredient that may impart either a functional (e.g., injectability, suspension, stability enhancing, drug release rate controlling) and/or a non-functional (e.g., processing aid or diluent) characteristic to the pharmaceutical composition. Suitable excipients for intravitreal injection compositions include, for example, buffering agents, stabilizing agents/surfactants, tonicity agents, pH adjusters, and cell targeting agents.

In some embodiments, the pharmaceutical composition comprises the carbon nanotubes in an about of about 0.5 mg/mL to about 1 mg/mL or more. For example, in some embodiments, the pharmaceutical composition comprises the carbon nanotubes in an amount of about 0.5 mg/mL, 0.6 mg/mL, 0.7 mg/mL, 0.8 mg/mL, 0.9 mg/mL, or 1.0 mg/mL, or more.

For example, the composition may include a buffer. Suitable buffers include, e.g., a phosphate buffer, an acetate buffer, a carbonate buffer, a citrate buffer, a histidine buffer, a lactate buffer, a succinate buffer, and a tartrate buffer. The pH of the buffer will typically be between about 2 to about 10, e.g., about pH 2, 3, 4, 5, 6, 7, 8, 9 or 10, e.g., about 4 to about 8. The pH can be adjusted using an acid or a base, for example, hydrochloric acid or sodium hydroxide.

The composition may include a stabilizing agent or surfactant, such as mannitol, sucrose, fructose, trehalose, lactose, histidine, lysine, glycine, cetrimide, docusate sodium, glyceryl monooleate, sodium lauryl sulfate, a sorbitan ester, or a mixture thereof. In some embodiments, the stabilizing agent or surfactant is a non-ionic surfactant. Suitable non-ionic surfactants include carboxylic esters, polyethylene glycol esters, glycol esters of fatty acids, ethoxylated aliphatic alcohols, cellulose derivatives (e.g., carboxymethylcellulose), polyoxyethylene surfactants, sorbitol esters, ethoxylated derivatives of sorbitol esters, glycol esters of fatty acids, and poloxamers.

The composition may include an ionic or non-ionic tonicity agent, e.g. glycerin, a sugar (including glucose, mannitol, sorbitol, trehalose, dextrose, lactose etc.), or a salt such as sodium chloride, potassium chloride, magnesium chloride, calcium chloride, sodium acetate, or the like. The use of a tonicity agent allows for control of the osmolality of the composition. For example, it may be desirable that a composition for intravitreal injection is isotonic to the vitreous so as to not disrupt the fluid balance of the vitreous and surrounding tissues. The compositions may have an osmolality of from about 250 to about 350 mOsmol/kg. For example, the compositions may have an osmolality of 250, 260, 270, 280, 290, 300, 310, 320, 330, 340 or 350 mOsmol/kg. One skilled in the art will appreciate that the amount of tonicity agent may vary depending on the particular choice of agent and on the other components in the composition.

The composition may include an antioxidant, such as ascorbic acid, sodium bisulfite, butylated hydroxyanisole, butylated hydroxytoluene, cysteine, cysteinate HCl, dithionite sodium, gentisic acid, gentisic acid ethanolamine, glutamate monosodium, formaldehyde sulfoxylate sodium, metabisulfite potassium, metabisulfite sodium, monothioglycerol, propyl gallate, sulfite sodium, or thioglycolate sodium. Alternatively, packaging may be configured in a manner that controls the potential for oxidation of the composition, including for example purging with an inert gas during manufacture.

In some embodiments, the pharmaceutical composition is provided in liquid form, e.g., as a solution in water. In other embodiments, the pharmaceutical composition is provided in solid form (e.g., a lyophilized mixture) that can be reconstituted with sterile water or aqueous solution prior to injection.

### Methods of Use

In some embodiments, disclosed herein are methods of treating an ocular tumor in a subject in need thereof, comprising injecting a composition into a vitreous body of the subject, wherein the composition comprises a complex of a carbon nanotube and a therapeutic agent (as described herein). In some embodiments, the composition is a pharmaceutical composition described herein, comprising the complex of the carbon nanotube and the therapeutic agent, and one or more pharmaceutically acceptable excipients.

In some embodiments, the ocular tumor is selected from retinoblastoma, uveal melanoma, and uveal metastasis. In some embodiments, the ocular tumor is retinoblastoma.

Retinoblastoma, a potentially deadly cancer, is the most common intraocular cancer of childhood. Within the last decade, the use of intraarterial and intravitreal chemotherapies significantly changed the management of retinoblastoma. Prior to intraarterial chemotherapy, systemic chemotherapy provided almost 100% globe salvage in group A, B, and C eyes when coupled with laser and cryotherapy and 48% in group D eyes (Shields et al. Arch Ophthalmol 1996, 114:1330-1338; Murphree et al. Arch Ophthalmol 1996, 114:1348-1356; Gallie et al. Arch Ophthalmol 1996, 114:1321-1328). The beneficial effect of intraarterial chemotherapy was more pronounced in group D eyes by improving the globe salvage rate up to 100% (Shields et al. Asia Pac J Ophthalmol 2016, 5:97-103; Manj andavida et al. Indian J Ophthalmol 2019, 67:740-754; Munier et al. Br J Ophthalmol 2017, 101:1086-1093; Abramson et al. Br J Ophthalmol 2012, 96:499-502). However, advanced group D eyes with vitreous seeds and group E eyes continued to be a challenging problem. The use of intravitreal injection especially improved the globe salvage rate in group D eyes with extensive vitreous seeds and group E eyes increasing the globe salvage rate from 27% to 73% (Shields et al. J Pediatr Ophthalmol Strabismus 2016, 53:275-284). The recurrence of main tumor rather than vitreous seeds was reported to be the reason of failure in globe salvage, suggesting the lack of penetration of chemotherapeutic agents in the main tumor (Ghassemi et al. Int Ophthalmol 2014, 34:533-540).

With the introduction of well-tolerated intravitreal injection technique in retinoblastoma, intravitreal chemotherapy gained an attention by improving the control of vitreous seeds which were the main reason of treatment failures (Francis et al. Neoplasia 2018, 20:757-763; Munier et al. Br J Ophthalmol 2012, 96:1078-1083; Shields et al. Curr Opin Ophthalmol 2014, 25:374-385). One study (Abramson et al. Br J Ophthalmol. 2019, 103(4):488-493) reviewed eyes treated with intravitreal chemotherapy for indications other than vitreous seeds including subretinal seeds and recurrent retinal tumors in 56 eyes of 52 patients and found the recurrence rate of retinal tumors in 19% of eyes and subretinal seeds in 11% of eyes. They concluded that intravitreal chemotherapy could be considered as adjuvant therapy in globe-sparing treatment. As disclosed in the examples herein, the penetration and diffusion of CNTs in retinoblastoma in animal models indicate that they can be used as an ideal carrier for chemotherapeutic agents in retinoblastoma, to provide treatment of vitreous or subretinal seeds or retinoblastoma.

The compositions disclosed herein can be administered to the eye via needle, or by a specialized delivery device for suprachoroidal space or intravitreal space. Specific methods for conducting an intravitreal injection are known to those skilled in the art. See, for example, Myers et al. Intravitreal Injection Technique: A Primer for Ophthalmology Residents and Fellows. EyeRounds.org. Jan. 6, 2015 (available from:

http://www.EyeRounds.org/tutorials/intravitreal-injection/).

For intravitreal injection, the composition can be administered in an amount of about 0.5 mL to about 1.0 mL per injection, e.g., about 0.5 mL, about 0.6 mL, about 0.7 mL, about 0.8 mL, about 0.9 mL, or about 1.0 mL.

In some embodiments, the methods of treating ocular tumors (e.g., retinoblastoma) further comprise one or more additional modes of chemotherapy, or other techniques such as surgery, radiation, laser therapy, cryotherapy, or photothermal therapy. For example, carbon nanotubes can absorb light in the near infrared (NIR) region, which can be exploited as a method to kill cancer cells via thermal effects. Accordingly, in some embodiments, the methods described herein are used in conjunction with photothermal therapy.

In some embodiments, disclosed herein are methods of detecting an ocular tumor in a subject, the method comprising: injecting a composition into a vitreous body of the subject, wherein the composition comprises a complex of a carbon nanotube and a diagnostic agent; and detecting a signal from the diagnostic agent. In some embodiments, the ocular tumor is selected from retinoblastoma, uveal melanoma, and uveal metastasis. In some embodiments, the ocular tumor is retinoblastoma. In some embodiments, the composition is a pharmaceutical composition described herein, comprising the complex of the carbon nanotube and the therapeutic agent (e.g., chemotherapeutic agent), and one or more pharmaceutically acceptable excipients. The detecting step will depend on the choice of diagnostic agent. In some embodiments, the detecting step comprises detection of a fluorescent, chromogenic, luminescent, or radioactive signal. In some embodiments, the detecting step comprises an imaging step such as positron emission tomography (PET), computed tomography (CT), single photon emission computerized tomography (SPECT), magnetic resonance imaging (MRI), and the like.

In some embodiments, disclosed herein is a use of a carbon nanotube for administration of a therapeutic agent (e.g., chemotherapeutic agent) or diagnostic agent into a vitreous body.

### Kits

In some embodiments, kits are provided that contain one or more or all of the components necessary, sufficient, or useful for practicing the methods described herein. In some embodiments, the kits comprise carbon nanotubes complexed to a therapeutic agent (e.g., chemotherapeutic agent) and/or diagnostic agent. In some embodiments, the kits comprise positive and/or negative control reagents. In some embodiments, the kits comprise instructions, which may be written instructions or embodied in a computer readable media. Reagents within the kits may be housed in one or more containers (e.g., tubes) and the collection of kit components may be packaged in one or more boxes or other containers that facilitate shipment and storage of the kit.

The following examples further illustrate aspects of the disclosure but, of course, should not be construed as in any way limiting its scope.

### EXAMPLES

All experiments are performed in accordance with the Association for Research in Vision and Ophthalmology (ARVO) statement for the Use of Animals in Ophthalmic and Visual Research. The protocol was approved by the University Committee on Use and Care of Animals of the University of Michigan. All surgeries were performed under ketamine and xylazine anesthesia, and all efforts were made to minimize suffering. The LH_{BETA}T_{AG} transgenic mice retinoblastoma animal model was used at 8-10 weeks old. Eye tumors in the LH_{BETA}T_{AG} transgenic mouse model showed the histological features of human retinoblastoma with endophytic and exophytic growth with invasion of retina, choroid and optic nerve (Albert et al. Trans. Am. Ophthalmol. Soc. 1994; 92:385-400). This animal model has been extensively characterized and develops bilateral multifocal retinal tumors that are stable and grow at the predictable rate (id.). In this model, retinoblastoma develops when the mouse is about 6 weeks old. When the mouse reaches the age of 8-10 weeks old, about half of the globe is filled with the tumor, and about at the age of 12-14 weeks, the entire globe is filed with retinoblastoma. Examination of each mouse was performed and it was confirmed that retinoblastoma tumor fills about 50% of the globe. This animal model was used as an example of a retinoblastoma intraocular tumor; the methods can also be used with other ocular tumors.

### Example 1

### Preparation of Targeted Carbon Nanotubes Functionalized with Fluorescein Isothiocyanate and Biotin (CNT-FITC-Bio), and Fluorescein Isothiocyanate and Folic Acid (CNT-FITC-FA)

CNTs were targeted by covalent attachment of biotin and folic acid. Receptors for biotin and folic acid are higher overexpressed on retinoblastoma cells than retinal pigment epithelium I (Jwala et al. J. Ocul. Pharmacol. Ther. 2012; 28:237-44). They were also attached with FITC to image them and to evaluate their penetration. In short, 0.5 mg CNTs with an average diameter of 1.2 nm and a length of 1000 nm (0.5 mg/mL, P3SWNT with 1.0-3.0 atomic % carboxylic acid, Carbon Solutions, Inc.) were dispersed in phosphate-buffered saline (PBS) buffer followed by incubation with 8 mg of 1-ethyl-3-(3-(dimethylamino)propyl) carbodiimide (EDAC) for 1 min at room temperature, after which samples were immediately vortexed. Next, Biotin and FITC (Life Technologies, CA) (2 μg in 20 μL of dimethylformamide) were added together, and the resulting mixture was allowed to react for an additional 2 h at 37° C. in a rotator rocker. These samples were then washed by PBS and centrifuged at 1300 rpm for 20 min for three times to remove unbound antibodies and excess FITC in Centricon YM-50 tubes (MilliporeSigma, MA), and the resulting CNT-FITC-Bio were suspended in 1 mL of serum-free Eagle's Minimum Essential Medium (EMEM) and used immediately. A similar procedure was applied to prepare CNTs functionalized with folic acid to prepare CNT-FITC-FA.

### Example 2

### CNT Injection and Analyses

Surgical Technique. 1 μl of 0.5 mg/mL targeted CNT-FITC-Bio, and CNT-FITC-FA were injected into the vitreous of one eye of LH_{BETA}T_{AG} transgenic mice. The other eye was not injected and was used as control eye. In each group of CNT-FITC-Bio (9 eyes) and CNT-FITC-FA (9 eyes), nine eyes of nine mice were used. The control group had the other uninjected nine eyes of nine mice. Vitreous injections were performed by an experienced team member (CL) under direct visualization with operating microscope, after dilating the pupil and confirming that injection was into the vitreous cavity, but not into tumor. During the procedure, the tip of the needle was constantly monitored. Three mice were sacrificed at each day 1, 2 and 3, and eyes were enucleated.

Histopathological Preparation. Mice eyes were fixated with 10% formaldehyde in phosphate-buffered saline (PBS) for histology or with 4% paraformaldehyde in phosphate-buffered saline followed by incubating with 30% sucrose in PBS. Eyes were embedded in optimal cutting temperature (OCT) compound and cryosectioned. They were stained with 4, diamidino-2-phenylindole (DAPI, 1 mg/mL in PBS; Sigma-Aldrich) to visualize cell nuclei.

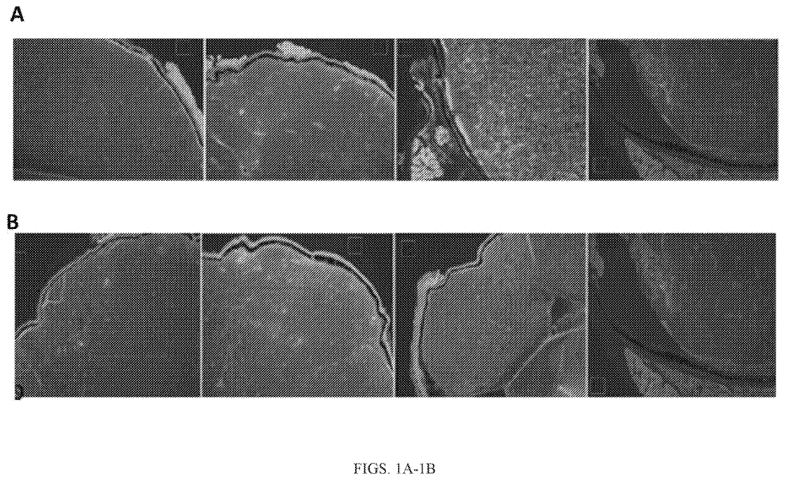

Image Analysis. Each globe was sectioned in to 5 μm thick sections. Five sections from each globe were evaluated and count of these five sections were averaged. The stained sections were imaged by Olympus BX-51 fluorescent microscope under 10× magnification. The fluorescent CNTs were excited by 480 nm light with same laser power and the camera exposure was kept the same all the time (which is 102.6 ms). The images were transformed into grey-scale images in MATLAB for intensity analysis. In order to compare the intensity, all the images were normalized. For normalization, a blue square region (blue square marked in FIGS. 1A and 1B) outside the eyeball area was marked in each eye, and the mean intensity within these same-sized blue squares were calculated for each eye. It is understandable that the glass region should reflect same, fixed light intensity. Therefore, all the images were normalized by setting the mean intensity of these blue squares to be the same. Finally, the fluorescent intensity for each image was calculated by calculating the intensity of a same-sized red square region (red squares marked in FIGS. 1A and 1B) within the eyeball area. The muscle cell area was avoided, which could also show strong fluorescents. The size of this square was determined by the largest possible area among the images. In order to compensate the false-positive results resulting from the photoconversion of DAPI due to blue excitation and green emission as reported (Jez et al. Histochem. Cell Biol. 2013, 139(1), 195-204), both control and injected eyes were analyzed and compared with each other.

Statistical Analysis. Difference in the fluorescein intensity between eyes injected with CNT-FITC-Bio and CNT-FITC-FA, and control eyes were compared by using Mann-Whitney U test. Similarly, difference in the fluorescein intensity between eyes injected with CNT-FITC-FA and CNT-FITC-Bio were compared by using Mann-Whitney U test. The change in the fluorescein intensity in eyes injected with CNT-FITC-FA and CNT-FITC-Bio and control eyes at different days were evaluated by using repeated measures ANOVA test.

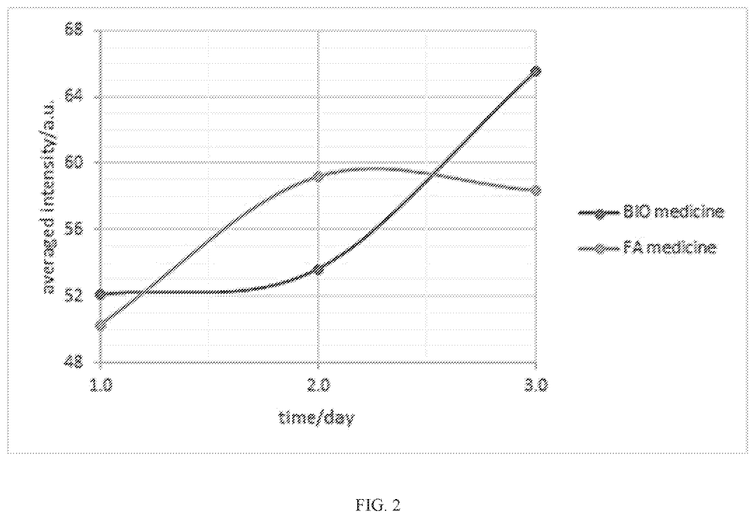

Results. Nine eyes of nine mice were included in each group of CNT-FITC-Bio and CNT-FITC-FA. The other uninjected nine eyes of nine mice were used as control. In all eyes of LH_{BETA}T_{AG} transgenic mice, retinoblastoma tumor occupied about 50% of mice eye. We found that the fluorescence intensity was higher in retinoblastoma tumor in eyes injected with CNT-FITC-FA and CNT-FITC-Bio than uninjected control eyes. (Table 1) The fluorescent intensity in the retinoblastoma tumor remained about the same on days 1 and 2, and mildly increased in day 3 for CNT-FITC-Bio. FIG. 1A) The fluorescent intensity increased on day 2 and remained about the same in day 3 for CNT-FITC-FA. (FIG. 2) We also observed that both CNT-FITC-Bio and CNT-FITC-FA passed through the retinoblastoma and stained the retinal pigment epithelium, showing their penetration through the tumor (FIG. 1B). The mean fluorescein intensity was significantly higher in eyes injected with CNT-FITC-Bio and CNT-FITC-FA compared to uninjected control eyes (p=0.02). We did not observe any difference in the mean fluorescein intensity between CNT-FITC-Bio and CNT-FITC-FA groups (p>0.05). There was no significant change in fluorescein intensity at different days in eyes injected with CNT-FITC-Bio and CNT-FITC-FA and uninjected control eyes. (p>0.05).

TABLE 1
Averaged intensities measured in eyes injected with CNT-FITC-Bio and CNT-FITC-FA

| | Day1 | Day2 | Day3 | Control |
|---|---|---|---|---|
| Averaged intensity CNT- | 52.08 ± 6.33 | 53.62 ± 9.00 | 65.54 ± 5.14 | 34.47 ± 6.67 |
| FITC-Bio sample/a.u. | (52, 42.60-56.55) | (53, 47.25-60) | (62, 58.26-84.25) | (34, 29.76-39.19) |
| Averaged intensity CNT- | 50.28 ± 7.37 | 59.21 ± 6.43 | 58.38 ± 2.32 | 34.47 ± 6.67 |
| FITC-FA sample/a.u. | (50, 45.07-55.49) | (55, 50.11-73.19) | (58, 56.74-60.01) | (34, 29.76-39.19) |

*The fluorescein intensity was significantly higher in eyes injected with CNT-FITC-Bio and CNT-FITC-FA compared to the control uninjected eyes (p = 0.02).
**There was no difference in fluorescein intensity between CNT-FITC-Bio and CNT-FITC-FA groups (p > 0.05).
***There was no significant change in fluorescein intensities at different days in the eyes with CNT-FITC-Bio and CNT-FITC-FA and the uninjected control eyes (p > 0.05).

Besides the tumor, staining in the lens, iris or cornea were not observed. There were no dose- or procedure-related complications (lens hit, cataract or retinal detachment).

Discussion. In this study, intravitreal CNTs penetrated and passed through the retinoblastoma tumor and reached the retinal pigment epithelium layer. Accordingly, CNTs can penetrate and diffuse through a solid tumor such as retinoblastoma when injected intravitreally. There was no difference in tumor penetration between the CNT-FITC-Bio and CNT-FITC-FA groups. The deep penetration into the tumor with the ability of CNTs to carry chemotherapeutic agents make them promising carrier candidates for both diagnostics and treatment of these hard-to-reach intraocular tumors.

1. A method of treating an ocular tumor in a subject in need thereof, comprising:
injecting a composition into a vitreous body of the subject, wherein the composition comprises a complex of a carbon nanotube and a therapeutic agent.

2. The method of claim 1, wherein the therapeutic agent is covalently bound to the carbon nanotube.

3. The method of claim 1, wherein the therapeutic agent is non-covalently bound to the carbon nanotube.

4. The method of any one of claims 1-3, wherein the therapeutic agent is a chemotherapeutic agent.

5. The method of any one of claims 1-4, wherein the complex further comprises one or more additional therapeutic agents.

6. The method of any one of claims 1-5, wherein the complex further comprises one or more additional agents selected from the group consisting of detectable moieties, targeting ligands, polymers, surfactants, nanoparticles, DNA, sugars, and lipids.

7. The method of claim 6, wherein the complex further comprises one or more targeting ligands selected from the group consisting of proteins, antibodies, peptides, metabolites, and receptors.

8. The method of any one of claims 1-7, wherein the composition further comprises a pharmaceutically acceptable excipient.

9. The method of any one of claims 1-8, wherein the carbon nanotube is a single-walled carbon nanotube.

10. The method of any one of claims 1-9, wherein the composition comprises a plurality of carbon nanotubes having an average diameter of 0.5 nm to 20 nm.

11. The method of any one of claims 1-10, wherein the composition comprises a plurality of carbon nanotubes having an average length of 50 nm to 5000 nm.

12. The method of any one of claims 1-11, wherein the ocular tumor is retinoblastoma.

13. A method of detecting an ocular tumor in a subject, the method comprising:
injecting a composition into a vitreous body of the subject, wherein the composition comprises a complex of a carbon nanotube and a diagnostic agent; and
detecting a signal from the diagnostic agent.

14. The method of claim 13, wherein the diagnostic agent is covalently bound to the carbon nanotube.

15. The method of claim 13, wherein the diagnostic agent is non-covalently bound to the carbon nanotube.

16. The method of any one of claims 13-15, wherein the complex further comprises one or more additional agents selected from the group consisting of therapeutic agents, targeting ligands, polymers, surfactants, nanoparticles, DNA, sugars, and lipids.

17. The method of claim 16, wherein the complex further comprises one or more targeting ligands selected from the group consisting of proteins, antibodies, peptides, metabolites, and receptors.

18. The method of any one of claims 13-17, wherein the composition further comprises a pharmaceutically acceptable excipient.

19. The method of any one of claims 13-18, wherein the carbon nanotube is a single-walled carbon nanotube.

20. The method of any one of claims 13-19, wherein the composition comprises a plurality of carbon nanotubes having an average diameter of 0.5 nm to 20 nm.

21. The method of any one of claims 13-20, wherein the composition comprises a plurality of carbon nanotubes having an average length of 50 nm to 5000 nm.

22. The method of any one of claims 13-21, wherein the ocular tumor is a retinoblastoma tumor.

23. The method of any one of claims 1-22, wherein the diagnostic agent is a fluorophore or a terahertz or sub-terahertz contrast agent.

24. An injectable composition for intravitreal injection into a vitreous body of an eye, comprising:
a complex of a carbon nanotube and a therapeutic or diagnostic agent; and
a pharmaceutically acceptable excipient.

25. The composition of claim 24, wherein the complex comprises a carbon nanotube and a therapeutic agent, and the therapeutic agent is covalently bound to the carbon nanotube.

26. The composition of claim 24, wherein the complex comprises a carbon nanotube and a therapeutic agent, and the therapeutic agent is non-covalently bound to the carbon nanotube.

27. The composition of any one of claims 24-26, wherein the therapeutic agent is a chemotherapeutic agent.

28. The composition of claim 24, wherein the complex comprises a carbon nanotube and a diagnostic agent, and the diagnostic agent is covalently bound to the carbon nanotube.

29. The composition of claim 24, wherein the complex comprises a carbon nanotube and a diagnostic agent, and the diagnostic agent is non-covalently bound to the carbon nanotube.

30. The composition of any one of claims 24-29, wherein the complex further comprises one or more additional agents selected from the group consisting of therapeutic agents, diagnostic agents, targeting ligands, polymers, surfactants, nanoparticles, DNA, sugars, and lipids.

31. The composition of claim 30, wherein the complex further comprises one or more targeting ligands selected from the group consisting of proteins, antibodies, peptides, metabolites, and receptors.

32. The composition of any one of claims 24-31, wherein the carbon nanotube is a single-walled carbon nanotube.

33. The composition of any one of claims 24-32, wherein the composition comprises a plurality of carbon nanotubes having an average diameter of 0.5 nm to 20 nm.

35. The composition of any one of claims 24-33, wherein the composition comprises a plurality of carbon nanotubes having an average length of 50 nm to 5000 nm.

36. Use of a carbon nanotube for administration of a therapeutic or diagnostic agent into a vitreous body.
